(19) United States
(12) Patent Application Publication
(10) Pub. No.: US 2012/0258130 A1
(43) Pub. Date: Oct. 11, 2012

(54) VACCINATION REGIMEN FOR B-CELL VACCINES

(76) Inventors: Patrik Maurer, Winterthur (CH); Thomas Pfister, Bubendorf (CH); Martin F. Bachmann, Seuzach (CH); Philipp Müller, Giebenach (CH); Wolfgang A. Renner, Kilchberg (CH)

(21) Appl. No.: 13/292,579

(22) Filed: Nov. 9, 2011

Related U.S. Application Data
(63) Continuation of application No. 12/594,650, filed on Oct. 5, 2009, now abandoned.

(30) Foreign Application Priority Data
Apr. 20, 2007 (EP) 07106676.5

(51) Int. Cl. A61K 39/385; A61P 25/34; A61P 25/30

(52) U.S. Cl. 424/194.1; 424/196.11

(57) ABSTRACT

This invention relates to the field of vaccination and treatment or prevention diseases. In particular this invention relates to the treatment or prevention of diseases by inducing hapten-specific antibody responses in a vaccinated subject. The invention further provides a method for prevention or treatment of a disease by inducing hapten-specific antibodies in a subject comprising administering into said subject a composition comprising a virus-like particle of an RNA bacteriophage and at least one hapten linked thereto.

### FIELD OF THE INVENTION

This invention relates to the field of vaccination and treatment or prevention diseases. In particular this invention relates to the treatment or prevention of diseases by inducing hapten-specific antibody responses in a vaccinated subject. The invention further provides a method for prevention or treatment of a disease by inducing hapten-specific antibodies in a subject comprising administering into said subject a composition comprising a virus-like particle of an RNA bacteriophage and at least one hapten linked thereto.

### PRIOR ART

The concept behind vaccination against haptens, which includes the majority of drugs of abuse, is that the anti-drug of abuse antibodies, which do not cross the blood-brain barrier, bind the drug in the blood and thus reduce the amount and rate of drug entering the central nervous system. By lowering the rewards associated with drug use, the addicted individual should no longer be motivated to consume the drug.

Nicotine vaccines have been tested in humans. The recombinant nicotine—Pseudomonas aeruginosa exoprotein A conjugate NicVAX was tested in smokers which were injected four times with either 50 μg, 100 μg or 200 μg NicVAX or placebo (Hatsukami D K et al, Clin Pharmacol Ther 2005, 78:456-467) at weeks 0, 4, 8 and 26. One injection of NicVAX was not sufficient to induce anti-nicotine antibodies and nicotine-specific antibodies were detected only after the second injection. Peak anti-nicotine titers were reached only at week 10, two weeks after the third injection.

Efficacy of a nicotine vaccine in smoking cessation has been demonstrated in a double-blind, placebo-controlled phase II smoking cessation study (Cornuz J et al., Presented at ASCO, May 2005; Orlando Fla., Available at http://www.asco.org/ac/1,1003,_{—}12-002643-00_{—}18-0034-00_{—}19-0033424,00.asp. Accessed on Nov. 25, 2005). Moderate to heavy smokers of total 340 subjects were recruited and were immunized five times with 100 μg NicQb (represents for nicotine molecules linked to the virus-like particle of an RNA bacteriophage) formulated in Alum or placebo in monthly intervals. The target quit date was set to month 1 after primary immunization. Continuous abstinence between month 2 and month 6 was slightly higher in subjects treated with NicQb than for placebo-treated subjects (28% versus 21%), but the difference did not reach statistical significance. However when smokers were stratified according to their anti-nicotine antibody titers, a robust statistically significant effect of vaccination was observed. 159 smokers in the active arm were divided in three groups depending on their titers. 57% of smokers with the highest antibody responses were continuously abstinent compared to 31% of subjects who received placebo; and this effect was highly significant (p=0.004). In contrast, subjects with medium or low antibody levels did not reach significant abstinence. The increased continuous abstinence rate persisted up to one year and 42% of the subjects with high antibody titers had not relapsed compared to 21% in the placebo group (Cytos: Vaccine to treat nicotine addiction promotes 12 months continuous abstinence in subjects who achieve high antibody levels. Press Release: Company webpage, http://www.cytos.com/doc/Cytos_Press_E_{—}051117.pdf. Accessed on Nov. 25, 2005). These, data clearly demonstrated that a vaccine against nicotine can be efficacious for smoking cessation in humans when anti-nicotine antibody levels are high enough and the antibodies have a sufficient affinity for nicotine.

Thus, there is a need for methods to induce higher effective anti-nicotine antibody levels. This is usually achieved by increasing the vaccine doses or by addition of an adjuvant. In addition, subjects usually relapse early during smoking cessation efforts. Initial lapses usually occur a few days after the initiation of smoking cessation and most subjects relapse within the first 8 weeks. Therefore, it is desirable to induce high anti-nicotine antibody levels as early as possible.

In addition, antibodies have to be of sufficient affinity. For several haptens it has been described that 1-2 weeks after immunization only antibodies with low affinities (Kd=1000-10000 nM) were induced while antibodies isolated after a few months showed affinity maturation and thus high affinities with Kd=10-100 nM (Foote and Eisen, PNAS, Vol. 97(20) pp. 10679-81, 1995).

### SUMMARY OF THE INVENTION

We have surprisingly found new vaccination regimens for hapten vaccines, in particular for vaccine for the prevention and treatment of addiction to a drug of abuse. The present regimens of the invention allow higher antibody titer achievable in immunized subjects, compared to the prior art regimens with time interval between administrations of around 28 days. For example, in the regimens described in the example section, the achieved highest antibody titer doubled in the regimens of the present invention than prior art. Furthermore these inventive regimens lead to a high antibody response to be reached much earlier than prior art. Again the regimen settings in the example section showed the peak reaching time can be shortened at least by half compared to the regimen done by prior art. Despite the present accelerated vaccination regimens, we have surprisingly found that the antibody affinity from the present regimens and from the prior art regimens are both comparably high.

Thus the present invention provides a method for prevention or treatment of a disease by inducing hapten-specific antibodies in a human comprising administering into said human a composition comprising: (a) a virus-like particle of an RNA bacteriophage with at least one first attachment site; and (b) at least one hapten with at least one, preferably only one, second attachment site, wherein (a) and (b) are covalently linked through the at least one first and the at least one second attachment site, and wherein the administering into the human of the composition comprises at least a first, a second and a third administration into the human of the composition, wherein the time interval between the first administration and the second administration, and between the second administration and the third administration is at most 18 days.

In a preferred embodiment, the present invention provides a method for prevention or treatment of addiction to a drug of abuse by inducing the drug of abuse-specific antibodies in a human comprising administering into said human a composition comprising, consisting essentially of, or consisting of: (a) a virus-like particle of an RNA bacteriophage with at least one first attachment site; and (b) at least one drug of abuse with at least one, preferably only one, second attachment site, wherein (a) and (b) are covalently linked through the at least one first and the at least one second attachment site, and wherein the administering into the human of the composition comprises at least a first, a second and a third administration into the human of the composition, wherein the time interval between the first administration and the second administration, and between the second administration and the third administration is at most 18 days. In one preferred embodiment, the time interval is at most 14 days. In one further preferred embodiment, the time interval is 14 days or 7 days.

In one preferred embodiment, the method of the present invention comprises or consists of five administrations, wherein the time interval between all the administrations is 14 days or 7 days. In one preferred embodiment, the method of the present invention comprises or consists of five administrations, wherein the time interval between the first and the second, the second and the third is 7 days and the time interval between the third and the fourth and the fourth and the fifth is 14 days.

In one preferred embodiment, the composition administered by the method of the present invention further comprises an adjuvant, wherein said adjuvant is preferably an aluminum salt, preferably aluminum hydroxide.

In a further aspect, the present invention provides for the use of a composition comprising (a) a virus-like particle of an RNA bacteriophage with at least one first attachment site, preferably a virus-like particle of an RNA bacteriophage Qβ with at least one first attachment site; and (b) at least one hapten with at least one, preferably only one, second attachment site; and wherein (a) and (b) are covalently linked through the at least one first and the at least one second attachment site, for the manufacture of a medicament for prevention or treatment of a disease by inducing hapten-specific antibodies in a human, wherein said composition is to be administered into said human, and where in said administering of said composition into said human comprises at least a first, a second and a third administration of said composition into said human, wherein the time interval between said first administration and said second administration, and between said second administration and said third administration is at most 18 days.

In a still further aspect, the present invention provides for a composition for prevention or treatment of a disease by inducing hapten-specific antibodies in a human, wherein said composition comprises: (a) a virus-like particle of an RNA bacteriophage with at least one first attachment site, preferably a virus-like particle of an RNA bacteriophage Qβ with at least one first attachment site; and (b) at least one hapten with at least one, preferably only one, second attachment site, wherein (a) and (b) are covalently linked through the at least one first and the at least one second attachment site, wherein said composition is to be administered into said human, and wherein the administering of said composition into said human comprises at least a first, a second and a third administration of said composition into said human, wherein the time interval between said first administration and said second administration, and between said second administration and said third administration is at most 18 days.

In a further preferred embodiment, the present invention provides for the use of a composition comprising (a) a virus-like particle of an RNA bacteriophage with at least one first attachment site, preferably a virus-like particle of an RNA bacteriophage Qβ with at least one first attachment site; and (b) at least one drug of abuse with at least one, preferably only one, second attachment site; and wherein (a) and (b) are covalently linked through the at least one first and the at least one second attachment site, for the manufacture of a medicament for prevention or treatment of addiction to a drug of abuse by inducing the drug of abuse-specific antibodies in a human, wherein said composition is to be administered into said human, and wherein said administering of said composition into said human comprises at least a first, a second and a third administration of said composition into said human, wherein the time interval between said first administration and said second administration, and between said second administration and said third administration is at most 18 days. In one further preferred embodiment, the time interval is at most 14 days, or further preferably, the time interval is 14 days. In a very preferred embodiment, the time interval is 7 days. In an again very preferred embodiment of the present invention, the use of said composition for the manufacture of a medicament for prevention or treatment of addiction to a drug of abuse further comprises or consists of a fourth administration. Further preferably, the time interval between the third administration and the fourth administration is at most 14 days, again further preferably the time interval between the third administration and the fourth administration is 14 days, and very preferably the time interval between the third administration and the fourth administration is 7 days. In an again further preferred embodiment of the present invention, the use of said composition for the manufacture of a medicament for prevention or treatment of addiction to a drug of abuse further comprises or consists of a fifth administration. Further preferably, the time interval between the first and the second, the second and the third, the third and the fourth is 7 days and between the fourth and the fifth is at least 14 days, preferably is 14 days, more preferably is 21 days and still more preferably is 28 days.

In a still further preferred embodiment, the present invention provides for a composition for prevention or treatment of addiction to a drug of abuse by inducing the drug of abuse-specific antibodies in a human, wherein said composition comprises: (a) a virus-like particle of an RNA bacteriophage with at least one first attachment site, preferably a virus-like particle of an RNA bacteriophage Qβ with at least one first attachment site; and (b) at least one drug of abuse with at least one, preferably only one, second attachment site, wherein (a) and (b) are covalently linked through the at least one first and the at least one second attachment site, wherein said composition is to be administered into said human, and wherein said administering of said composition into said human comprises at least a first, a second and a third administration of said composition into said human, wherein the time interval between said first administration and said second administration, and between said second administration and said third administration is at most 18 days. In one further preferred embodiment, the time interval is at most 14 days, or further preferably, the time interval is 14 days. In a very preferred embodiment, the time interval is 7 days. In an again very preferred embodiment of the present invention, the composition for prevention or treatment of addiction to a drug of abuse further comprises or consists of a fourth administration of said composition to said human. Further preferably, the time interval between the third administration and the fourth administration is at most 14 days, again further preferably the time interval between the third administration and the fourth administration is 14 days, and very preferably the time interval between the third administration and the fourth administration is 7 days. In an again very preferred embodiment of the present invention, the composition for prevention or treatment of addiction to a drug of abuse further comprises or consists of a fifth administration. Further preferably, the time interval between the first and the second, the second and the third, the third and the fourth is 7 days and between the fourth and the fifth is at least 14 days, preferably is 14 days, more preferably is 21 days and still more preferably is 28 days.

In again another further preferred embodiment, the present invention provides for a composition for prevention or treatment of addiction to nicotine by inducing the nicotine-specific antibodies in a human, wherein said composition comprises: (a) a virus-like particle of an RNA bacteriophage Q and (b) at least one nicotine molecule, wherein the at least one nicotine molecule is covalently bound to the virus-like particle by a linking sequence, wherein the linking sequence consists of A-CH2OCO(CH2)2CO—B, and wherein A represents said nicotine molecule and wherein B represents said virus-like particle of RNA bacteriophage Q, and wherein said linking sequence is covalently bound to the 3′ position of said nicotine molecule, and wherein said composition is to be administered into said human, and wherein said administering of said composition into said human comprises at least a first, a second and a third administration of said composition into said human, wherein the time interval between said first administration and said second administration, and between said second administration and said third administration is 7 days. Further preferably, this composition for prevention or treatment of addiction to a drug of abuse further comprises a fourth administration of said composition to said human, wherein preferably, the time interval between the third administration and the fourth administration is 7 days. Again more preferably, the composition for prevention or treatment of addiction to a drug of abuse further comprises a fifth administration, wherein preferably, the time interval between the fourth and the fifth is at least 14 days, preferably is 14 days, more preferably is 21 days and still more preferably is 28 days.

In a further aspect, the present invention provides for a composition for prevention or treatment of addiction to nicotine by inducing the nicotine-specific antibodies in a human, wherein said composition comprises: (a) a virus-like particle of an RNA bacteriophage Qβ and (b) at least one nicotine molecule, wherein the at least one nicotine molecule is covalently bound to the virus-like particle by a linking sequence, wherein the linking sequence consists of A-CH₂OCO(CH₂)₂CO—B, and wherein A represents said nicotine molecule and wherein B represents said virus-like particle of RNA bacteriophage Qβ, and wherein said linking sequence is covalently bound to the 3′ position of said nicotine molecule, and wherein said composition is formulated for administering into said human, and wherein said administering of said composition into said human comprises at least a first, a second and a third administration of said composition into said human, wherein the time interval between said first administration and said second administration, and between said second administration and said third administration is 7 days. Further preferably, this composition for prevention or treatment of addiction to a drug of abuse further comprises a fourth administration of said composition to said human, wherein preferably, the time interval between the third administration and the fourth administration is 7 days. Again more preferably, the composition for prevention or treatment of addiction to a drug of abuse further comprises a fifth administration, wherein preferably, the time interval between the fourth and the fifth is at least 14 days, preferably is 14 days, more preferably is 21 days and still more preferably is 28 days.

In a further aspect, the present invention provides for a composition for prevention or treatment of addiction to nicotine by inducing the nicotine-specific antibodies in a human, wherein said composition comprises: (a) a virus-like particle of an RNA bacteriophage Qβ and (b) at least one nicotine molecule, wherein the at least one nicotine molecule is covalently bound to the virus-like particle by a linking sequence, wherein the linking sequence consists of A-CH₂OCO(CH₂)₂CO—B, and wherein A represents said nicotine molecule and wherein B represents said virus-like particle of RNA bacteriophage Qβ, and wherein said linking sequence is covalently bound to the 3′ position of said nicotine molecule, and wherein said composition is adapted or customized for administering into said human, and wherein said administering of said composition into said human comprises at least a first, a second and a third administration of said composition into said human, wherein the time interval between said first administration and said second administration, and between said second administration and said third administration is 7 days. Further preferably, this composition for prevention or treatment of addiction to a drug of abuse further comprises a fourth administration of said composition to said human, wherein preferably, the time interval between the third administration and the fourth administration is 7 days. Again more preferably, the composition for prevention or treatment of addiction to a drug of abuse further comprises a fifth administration, wherein preferably, the time interval between the fourth and the fifth is at least 14 days, preferably is 14 days, more preferably is 21 days and still more preferably is 28 days.

### BRIEF DESCRIPTION OF THE FIGURES

FIG. 1: Comparison of vaccination regimens 1, 2 and 3: Subjects were immunized with 100 μg NicQb in the presence of Alum either 5 times in weekly intervals (closed diamonds, regimen 3), 5 times in biweekly intervals (open squares, regimen 2) or five times with a monthly interval (crosses, regimen 1). Nicotine-specific IgG titers were determined for all subjects by ELISA and geometric mean ELISA titers of the three groups are shown.

FIG. 2: A) Comparison of vaccination regimens 1 and 4: Subjects were immunized with 100 μg NicQb in the presence of Alum either five times with a monthly interval (crosses, regimen 1) or with four injections at weekly intervals followed by one injection 28 days later (regimen 4). B) Comparison of vaccination regimens 1 and 5: Subjects were immunized with 100 μg NicQb in the presence of Alum either five times with a monthly interval (crosses, regimen 1) or with three injections at weekly intervals followed by two injections at monthly intervals (regimen 5). Nicotine-specific IgG titers were determined for all subjects by ELISA and geometric mean ELISA titers of the groups are shown.

### DETAILED DESCRIPTION OF THE INVENTION

Unless defined otherwise, all technical and scientific terms used herein have the same meanings as commonly understood by one of ordinary skill in the art to which this invention belongs.

Adjuvant: The term “adjuvant” as used herein refers to non-specific stimulators of the immune response or substances that allow generation of a depot in the host which when combined with the composition, vaccine composition or pharmaceutical composition of the invention provides for an even more enhanced and/or prolonged immune response, preferably cytokine production. A variety of adjuvants is known in the art and useful in the invention. Preferred adjuvants are selected from the group consisting of complete or incomplete Freund's adjuvant, aluminum containing adjuvants, modified muramyldipeptide, surface active substances such as lysolecithin, pluronic polyols, polyanions, peptides, oil emulsions, keyhole limpet hemocyanins, dinitrophenol, BCG (bacille Calmette Guerin) Corynebacterium parvum, ligands of toll-like receptors (TLR) which include but are not limited to peptidoglycans, lipopolysaccharides and their derivatives, poly I:C, immunostimulatory oligonucleotides, imidazoquinolines such as resiquimod and imiquimod, flagellins, monophosphoryl lipid immunomodulator, AdjuVax 100a, QS-21, QS-18, GPI-0100, CRL1005, MF-59, OM-174, OM-197, OM-294, Virosomal adjuvant technology and any mixture thereof. A very preferred adjuvant for the purpose of the invention is aluminum containing adjuvant, preferably an aluminum salt, preferably aluminum hydroxide, preferably an aluminum containing mineral gel, most preferably alhydrogel. In a very preferred embodiment said adjuvant is alhydrogel. The term adjuvant also encompasses a mixture of any of the substances listed above. Particles of the invention, preferably VLPs, have been generally described as an adjuvant. However, the term “adjuvant”, as used within the context of this application, refers to an adjuvant not being the VLP of RNA-bacteriophage used for the inventive compositions. In each case, the term adjuvant refers to an adjuvant used in addition to the VLP.

Attachment Site, First: As used herein, the term “first attachment site” refers to an element which is naturally occurring with the VLP of RNA-bacteriophage or which is artificially added to the VLP of RNA-bacteriophage, and to which the second attachment site may be linked The first attachment site may be a protein, a polypeptide, an amino acid, a peptide, a sugar, a polynucleotide, a natural or synthetic polymer, a secondary metabolite or compound (biotin, fluorescein, retinol, digoxigenin, metal ions, phenylmethylsulfonylfluoride), or a chemically reactive group such as an amino group, a carboxyl group, a sulfhydryl group, a hydroxyl group, a guanidinyl group, histidinyl group, or a combination thereof. A preferred embodiment of a chemically reactive group being the first attachment site is the amino group of an amino acid such as lysine. The first attachment site is located, typically on the surface, and preferably on the outer surface of the VLP. Multiple first attachment sites are present on the surface, preferably on the outer surface of virus-like particle of an RNA bacteriophage, typically in a repetitive configuration.

Attachment Site, Second: As used herein, the term “second attachment site” refers to an element part of or associated with the hapten, or preferably the drug of abuse, to which the first attachment site on the surface of the VLP of RNA bacteriophage may associate. The second attachment site of the hapten, or preferably of the drug of abuse, comprises any chemical moiety, preferably a amine, an amide, a carboxyl, a sulfhydryl, hydroxyl, aldehyde, acylhalogenide, hydrazine, diazonium, or hydrazide, or further chemical moieties able to specifically react with the first attachment site. At least one, preferably only one, second attachment site is present on the hapten or preferably the drug of abuse. The term “hapten” or preferably “drug of abuse with at least one second attachment site” refers, therefore, to a hapten, or preferably a “drug of abuse”, construct comprising at least the hapten, or preferably the “drug of abuse”, and the second attachment site. However, in particular for a second attachment site, which is not naturally occurring within the hapten or preferably the drug of abuse, these haptens or drugs of abuse comprise a linker which associates the hapten or the drug of abuse with the second attachment site, or more preferably, already comprises or contains the second attachment site.

Coat protein: The term “coat protein” and the interchangeably used term “capsid protein” within this application, refers to a viral protein, preferably a subunit of an RNA bacteriophage, which is capable of assembling into a virus capsid or a VLP of an RNA bacteriophage.

Hapten: As used herein, the term “hapten” refers to a low-molecular weight organic compound that is not capable of eliciting an immune response by itself but will elicit an immune response once attached to a carrier molecule. Exemplary haptens used in conjugates, compositions and methods of the invention include drugs, hormones and toxins, but are not limited to these specific haptens. A majority of drug of abuse are haptens.

Linked: The term “linked” (or its noun: linkage) as used herein, refers to all possible ways, preferably chemical interactions, by which the at least one first attachment site and the at least one second attachment site are joined together. Chemical interactions include covalent and non-covalent interactions. Typical examples for non-covalent interactions are ionic interactions, hydrophobic interactions or hydrogen bonds, whereas covalent interactions are based, by way of example, on covalent bonds such as ester, ether, phosphoester, amide, peptide, carbon-phosphorus bonds, carbon-sulfur bonds such as thioether, or imide bonds. In certain preferred embodiments the first attachment site and the second attachment site are linked through at least one covalent bond. The term “linked” as used herein, however, shall not only encompass a direct linkage of the at least one first attachment site and the at least one second attachment site but also, alternatively and preferably, an indirect linkage of the at least one first attachment site and the at least one second attachment site through intermediate molecule(s), and hereby typically and preferably by using at least one, preferably one, heterobifunctional cross-linker.

Nicotine: The term “nicotine” as used in the present invention refers to nicotine, either in its enantiomerically pure (S)- or (R)-form or a mixture thereof, which could be derivatized in such manner as to contain at least one second attachment site which, then, is capable of associating with the first attachment site of the carrier either directly, or via a cross-linker. Preferably, the nicotine is derivatized in such manner as to contain only one second attachment site.

Virus-like particle of a RNA phage: As used herein, the term “virus-like particle of a RNA phage” refers to a virus-like particle comprising, or preferably consisting essentially of or consisting of coat proteins, mutants or fragments thereof, of a RNA phage. In addition, virus-like particle of a RNA phage resembling the structure of a RNA phage, being non replicative or non-infectious, and typically and preferably being non replicative and non-infectious. Typically and preferably, the term “virus-like particle of a RNA phage” should furthermore refer to a virus-like particle of a RNA phage which lacks at least one of the genes, preferably all of the genes, encoding for the replication machinery of the RNA phage, and typically and further preferably even at least one of the genes, preferably all of the genes, encoding the protein or proteins responsible for viral attachment to or entry into the host. This definition should, however, also encompass virus-like particles of RNA phages, in which the aforementioned gene or genes are still present but inactive, and, therefore, also leading to non-replicative and/or noninfectious virus-like particles of a RNA phage. Moreover, the term “virus-like particle of a RNA phage” should therefore also encompass in its broadest definition a virus particle of a RNA phage, the genome of which has been inactivated by physical or chemical or genetic methods so that the virus particle is not capable of infecting and/or replicating. Preferred VLPs derived from RNA-phages exhibit icosahedral symmetry and consist of 180 subunits. Within this present disclosure the term “subunit” and “monomer” are interexchangeably and equivalently used within this context. In this application, the term “RNA-phage” and the term “RNA-bacteriophage” are interchangeably used.

One, a, or an: when the terms “one”, “a”, or “an” are used in this disclosure, they mean “at least one” or “one or more” unless otherwise indicated.

The amino acid sequence identity of polypeptides can be determined conventionally using known computer programs such as the Bestfit program. When using Bestfit or any other sequence alignment program, preferably using Bestfit, to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed. This aforementioned method in determining the percentage of identity between polypeptides is applicable to all proteins, polypeptides or a fragment thereof disclosed in this invention.

It will be understood by one of ordinary skill in the relevant arts that other suitable modifications and adaptations to the methods and applications described herein are readily apparent and may be made without departing from the scope of the invention or any embodiment thereof. In particular in this application the time interval expressed by days, such as “at most 14 days”, “is 14 days”, “is 7 days”, is to be understood to have a derivation of plus or minus two days, preferably plus or minus one day. Other numerical values expressed herein, except the values experimentally determined within the examples, shall include the stated value and a value range of ±10% of the stated value.

In one aspect of the invention, the present invention provides a method for prevention or treatment of a disease by inducing hapten-specific antibodies in a human comprising administering into the human a composition comprising, consisting essentially of, or consisting of: (a) a virus-like particle of an RNA bacteriophage with at least one first attachment site; and (b) at least one hapten with at least one, preferably only one, second attachment site, wherein (a) and (b) are covalently linked through the at least one first and the at least one second attachment site, and wherein the administering into the human of the composition comprises at least a first, a second and a third administration into the human of the composition, wherein the time interval between the first administration and the second administration, and between the second administration and the third administration is at most 18 days.

In one aspect of the invention, the present invention provides a method for prevention or treatment of addiction to a drug of abuse by inducing the drug of abuse-specific antibodies in a human comprising administering into the human a composition comprising, consisting essentially of, or consisting of: (a) a virus-like particle of an RNA bacteriophage with at least one first attachment site; and (b) at least one drug of abuse with at least one, preferably only one, second attachment site, wherein (a) and (b) are covalently linked through the at least one first and the at least one second attachment site, and wherein the administering into the human of the composition comprises at least a first, a second and a third administration into the human of the composition, wherein the time interval between the first administration and the second administration, and between the second administration and the third administration is at most 18 days. Preferably the time interval between the first administration and the second administration, and between the second administration and the third administration is at least three days, preferably at least four days, more preferably at least five days. In one preferred embodiment, the time interval between the first administration and the second administration, and between the second administration and the third administration is at least five days and at most 18 days.

In one preferred embodiment, the time interval between the first administration and the second administration, and between the second administration and the third administration is at most 14 days, or at most 10, or preferably at most 7 days. Further preferably the time interval between the first administration and the second administration, and between the second administration and the third administration is least three days, preferably at least four days, more preferably at least five days. In one preferred embodiment, the time interval between the first administration and the second administration, and between the second administration and the third administration is at least five days and at most 14 days. In one preferred embodiment, the time interval between the first administration and the second administration, and between the second administration and the third administration is at least five days and at most 10 days. In one preferred embodiment, the time interval between the first administration and the second administration, and between the second administration and the third administration is at least five days and at most 7 days.

In one preferred embodiment, the time interval between the first administration and the second administration and the time interval between the second administration and the third administration are the same.

In a very preferred embodiment, the time interval between the first administration and the second administration, and between the second administration and the third administration is 14 days. In an alternatively very preferred embodiment of the present invention, the time interval between the first administration and the second administration, and between the second administration and the third administration is 7 days.

In one preferred embodiment, the method of the present invention further comprises further administrations. In one preferred embodiment, the method of the present invention comprises or consists of a further administration, or two further administrations or three administrations.

In one preferred embodiment, the method of present invention further comprises or consists of a fourth administration. In one preferred embodiment, the time interval between the third administration and the fourth administration is equal to the time interval between the first administration and the second administration. In one preferred embodiment, the time interval between the first and the second, the second and the third and the third and the fourth are the same. In another preferred embodiment, the time interval between the third administration and the fourth administration is longer than the time interval between the first administration and the second administration and is longer than the time interval between the second administration and the third administration. In one preferred embodiment, the time interval between the third administration and the fourth administration is twice as long as the time interval between the first administration and the second administration, and is twice as long as the time interval between the second administration and the third administration. In one preferred embodiment, the time interval between the third administration and the fourth administration is at most 14 days, preferably is 14 days or preferably is 7 days.

In one preferred embodiment, the method of present invention further comprises or consists of a fifth administration. In one preferred embodiment, the time interval between the fourth administration and the fifth administration is equal to the time interval between the first administration and the second administration. In another preferred embodiment, the time interval between the fourth administration and the fifth administration is longer than the time interval between the first administration and the second administration and is longer than the time interval between the second administration and the third administration. In one preferred embodiment, the time interval between the fourth administration and the fifth administration is twice as long as the time interval between the first administration and the second administration, and is twice as long as the time interval between the second administration and the third administration. In one preferred embodiment, the time interval between the third administration and the fourth administration and the time interval between the fourth administration and the fifth administration are the same. In one preferred embodiment, the time interval between the fourth administration and the fifth administration is at most 14 days, preferably is 14 days or preferably is 7 days.

In one preferred embodiment, the time interval between the first administration and the second, the second and the third, the third and the fourth and the fourth and the fifth are all the same. In one preferred embodiment, the time interval is at most 14 days. In one further preferred embodiment, the time interval is 14 days. In another further preferred embodiment, the time interval is 7 days.

In one preferred embodiment, the time interval between the third and the fourth and between the fourth and the fifth are longer than the time interval between the first and the second and the second and the third. In one preferred embodiment, the time interval between the first administration and the second, the second and the third is 7 days and the time interval between the third and the fourth and the fourth and the fifth is 14 days. In one preferred embodiment, the time interval between the first administration and the second, the second and the third is 14 days and the time interval between the third and the fourth and the fourth and the fifth is 28 days. The advantage of after the first three initial administrations, extend the time interval of the later administrations is to obtain a prolonged high antibody titer in the subject.

In one preferred embodiment, the time interval between the first and the second, the second and the third is 7 days and between the third and the fourth, the fourth and the fifth is at least 14 days, preferably is 14 days, more preferably is 21 days and still more preferably is 28 days.

In another preferred embodiment, the time interval between the first and the second, the second and the third, the third and the fourth is 7 days and between the fourth and the fifth is at least 14 days, preferably is 14 days, more preferably is 21 days and still more preferably is 28 days.

It has been generally believed that the maturation of the antibodies, i.e., the evolvement of high affinity antibodies towards an antigen takes some time, usually several months. However we have surprising found that with our accelerated vaccination regimens, i.e., the time interval between the first administration and the second, and between the second and the third is at most 14 days, preferably the time interval is 14 days or 7 days, or with our even more preferred regimens, i.e., the time intervals between the five administrations is at most 14 days, preferably is 14 days or 7 days, not only high antibody response can be achieved within shorter time than prior art, furthermore and more importantly the obtainable or obtained antibodies have a high affinity of Kd lower than 500 nM, which is comparable to prior art.

Thus in one preferred embodiment, the method of the present invention leads to the highest antibody response to be reached within 10 weeks, preferably within 4 weeks.

Furthermore in one preferred embodiment, the antibody specific for a hapten, preferably for a drug of abuse, more preferably for a nicotine molecule, obtainable or obtained from the present invention, has an affinity of which Kd is lower than 500 nM, preferably lower than 150 nM, more preferably lower than 100 nM, more preferably lower than 50 nM, more preferably lower than 10 nM, more preferably lower than 1 nM.

In one preferred embodiment, the RNA bacteriophage is selected from the group consisting of: a) bacteriophage Qβ; b) bacteriophage R17; c) bacteriophage fr; d) bacteriophage GA; e) bacteriophage SP; f) bacteriophage MS2; g) bacteriophage M11; h) bacteriophage MX1; i) bacteriophage NL95; k) bacteriophage f2; l) bacteriophage PP7 and m) bacteriophage AP205.

In one preferred embodiment, the virus-like particle of an RNA bacteriophage comprises, or alternatively consists essentially of, or consists of, coat proteins, recombinant coat proteins, mutants or fragments thereof, of an RNA bacteriophage. The term “fragment of a coat protein”, as used herein, is defined as a polypeptide, which is of at least 70%, preferably at least 80%, more preferably at least 90%, even more preferably at least 95% the length of the wild-type coat protein and which preferably retains the capability of forming VLP of an RNA bacteriophage. Preferably the fragment is obtained by at least one internal deletion, at least one truncation or at least one combination thereof. The term “fragment of a coat protein” shall further encompass polypeptide, which has at least 80%, preferably 90%, even more preferably 95% amino acid sequence identity with the “fragment of a coat protein”, as defined above and which is preferably capable of assembling into a virus-like particle of an RNA bacteriophage.

The term “mutant coat protein” or the term “mutant of a coat protein”, as interchangeably used in this invention, refers to a polypeptide having an amino acid sequence derived from the coat protein, wherein the amino acid sequence is at least 80%, preferably at least 85%, 90%, 95%, 97%, or 99% identical to the coat protein and preferably retains the ability to assemble into a VLP of an RNA bacteriophage.

In one preferred embodiment of the invention, the composition comprises coat protein, mutants or fragments thereof, of RNA phages, wherein the coat protein has amino acid sequence selected from the group consisting of: (a) SEQ ID NO:1; referring to Qβ CP; (b) a mixture of SEQ ID NO:1 and SEQ ID NO:2 (referring to Qβ A1 protein); (c) SEQ ID NO:3; (d) SEQ ID NO:4; (e) SEQ ID NO:5; (1) SEQ ID NO:6, (g) a mixture of SEQ ID NO:6 and SEQ ID NO:7; (h) SEQ ID NO:8; (i) SEQ ID NO:9; (j) SEQ ID NO:10; (k) SEQ ID NO:11; (l) SEQ ID NO:12; (m) SEQ ID NO:13; and (n) SEQ ID NO:14.

In one preferred embodiment, the RNA bacteriophage is Qβ. Particular example 18 of WO 02/056905 gave detailed description of preparation of VLP particles from Qβ.

Qβ mutant coat protein, of which exposed lysine residues are replaced by arginines can be used for the present invention. In a preferred embodiment, Qβ mutant coat protein is selected from the group consisting of: a) Qβ-240 (SEQ ID NO:15, Lys13-Arg of SEQ ID NO: 1) b) Qβ-243 (SEQ ID NO:16, Asn10-Lys of SEQ ID NO:1); c) Qβ-250 (SEQ ID NO:17, Lys2-Arg of SEQ ID NO:1) d) Qβ-251 (SEQ ID NO:18, Lys16-Arg of SEQ ID NO:1); and e) Qβ-259″ (SEQ ID NO:19, Lys2-Arg, Lys16-Arg of SEQ ID NO:1).

In one preferred embodiment, the RNA bacteriophage is AP205.

In another very preferred embodiment of the present invention, the virus-like particle of an RNA bacteriophage is a virus-like particle of RNA bacteriophage Qβ comprising coat proteins of RNA bacteriophage Qβ, wherein said coat proteins have the amino acid sequence of SEQ ID NO:1. In a further very preferred embodiment of the present invention, the virus-like particle of an RNA bacteriophage is a virus-like particle of RNA bacteriophage Qβ consisting essentially of or consisting of coat proteins of RNA bacteriophage Qβ, wherein said coat proteins have the amino acid sequence of SEQ ID NO:1.

In one preferred embodiment, the first attachment site comprises, or preferably is, an amino group, preferably the amino group of a lysine residue.

In one preferred embodiment, the drug of abuse is selected from the group consisting of: (a) codeine; (b) fentanyl; (c) heroin; (d) morphine; (e) amphetamine; (f) cocaine; (g) methylenedioxymethamphetamine; (h) methamphetamine; (i) methylphenidate; (j) nicotine; (k) cotinine; (l) nornicotine; (m) PCP; (n) LSD; (o) mescaline; (p) psilocybin; (q) tetrahydrocannabinol; (r) diazepam; (s) desipramine; (t) imipramine; (u) nortriptyline; and (v) the amitriptyline class of drugs.

In one preferred embodiment, the drug of abuse is nicotine. The totality of the covalently bound nicotine molecules are either present in the same absolute configuration, i.e. all nicotine molecules have the (R)-configuration or all nicotine molecules have the naturally occurring (S)-configuration, or they are present in any mixture thereof. Preferably, the nicotine molecules are covalently bound such as about an equal mixture or an equal mixture of both the (R)-configuration and the naturally occurring (S)-configuration is present. In a very preferred embodiment, the nicotine-VLP conjugate comprised by the inventive formulations is obtainable or obtained by using a racemic mixture of nicotine molecules or nicotine derivatives, typically and preferably by using a racemic mixture of nicotine molecules or nicotine derivatives comprising the nicotine molecules with a linking sequence covalently bound thereto, for the coupling reaction to the VLP of RNA bacteriophage.

In one preferred embodiment, the drug of abuse is nicotine metabolites including nornicotine and cotinine. Therapy for nicotine addiction may also target nicotine metabolites including nornicotine and cotinine, both of which have longer half lives than nicotine, have pharmacologic and neuropharmacologic affects similar to nicotine and may be addictive. U.S. Pat. No. 6,932,971, in particular from column 42 to 43, describes different sites of linkage and means of linkage of the nicotine or nicotine metabolites to the protein carrier.

In one preferred embodiment, the drug of abuse is cocaine, for example succinylated norcocaine. U.S. Pat. No. 6,932,971, in particular from column 43 to 45 till the second paragraph, describes different sites of linkage and means of linkage of the cocaine and related drugs to the protein carrier.

In one preferred embodiment, the drug of abuse is heroine.

Suitable second attachment site, which naturally occurs with or is derivable from the hapten, preferably the drug of abuse, are known to a skilled person. U.S. Pat. No. 6,932,971 describes, in particular, from column 37 to column 41, the nature, location and generation of second attachment site and these teachings are incorporated herein by way of reference. Chemical derivatization of a hapten, preferably a drug of abuse, leads to said haptens, or preferably said drug of abuse, comprises a linker which associates the hapten, or preferably drug of abuse, with the second attachment site, or more preferably, already comprises or contains the second attachment site.

In one very preferred embodiment of the present invention, the composition comprises (a) a virus-like particle of an RNA bacteriophage Qβ and (b) at least one nicotine molecule, wherein the at least one nicotine molecule is covalently bound to the virus-like particle by a linking sequence, wherein the linking sequence consists of A-CH₂OCO(CH₂)₂CO—B, and wherein A represents said nicotine molecule and wherein B represents said virus-like particle of RNA bacteriophage Qβ, and wherein said linking sequence is covalently bound to the 3′ position of said nicotine molecule.

In one preferred embodiment of the present invention, the composition further comprises an adjuvant, wherein preferably is aluminum containing adjuvant, preferably an aluminum salt, preferably aluminium hydroxide, preferably an aluminum containing mineral gel, most preferably alhydrogel. In one preferred embodiment of the present invention, the composition comprises from 1 mg to 2 mg, preferably from 1.2 mg to 1.7 mg, more preferably from 1.3 mg to 1.5 mg of aluminium salt, preferably aluminium hydroxide.

In one preferred embodiment, during each administration substantially the same dose of said composition is administered.

In one preferred embodiment, during each administration a dose of at least 50 μg of the composition, preferably at least 100 μg, or preferably at least 200 μg or at least 300 μg is administered. The dose of the administered composition shall not preferably exceed 500 μg, preferably not exceeds 400 μg. In one preferred embodiment of the present invention, during each administration about 100 μg of the composition is administered in a human.

The compositions of the present invention may be administered by various methods known in the art, but will normally be administered by injection, infusion, inhalation, oral administration, or other suitable physical methods. The compositions may alternatively be administered intramuscularly, intravenously, transmucosally, transdermally or subcutaneously. In one very preferred embodiment, the administration of the composition is administered subcutaneously. Components of compositions for administration include sterile aqueous (e.g., physiological saline) or non-aqueous solutions and suspensions. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption.

### EXAMPLE 1

“NicQβ”—The term “NicQβ”, as used herein should refer to at least one nicotine-VLP conjugate comprising (a) a virus-like particle of RNA bacteriophage Qβ; and (b) at least one nicotine molecule, wherein the nicotine molecule is covalently bound to VLP of Qβ by a linking sequence, wherein said linking sequence consists of A-CH₂OCO(CH₂)₂CO—B, and wherein A represents said nicotine molecule and wherein B represents the VLP of Qβ, and wherein the linking sequence is covalently bound to the 3′ position of the nicotine molecule. NicQβ was produced as described in EXAMPLE 1 of U.S. Pat. No. 6,932,971. NicQβ drug substance was thawed at room temperature (regimen 1) or reconstituted with sterile water from a freeze-dried powder followed by the addition of an adjuvant Alhydrogel™ containing aluminum hydroxide [Al(OH)₃].

The final vaccine composition ready for administration, designated as “100 μg-dose”, containing 100 μg of NicQβ and 1.3 mg of aluminum hydroxide.

### EXAMPLE 2

Regimen 1: Subject received five doses prepared as described in Example 1. The time interval between the administrations was 28 days

Regimen 2: Substantially the same as Regimen 1 with the exception that the time interval between the administrations was 14 days.

Regimen 3: Substantially the same as Regimen 1 with the exception that the time interval between the administrations was 7 days.

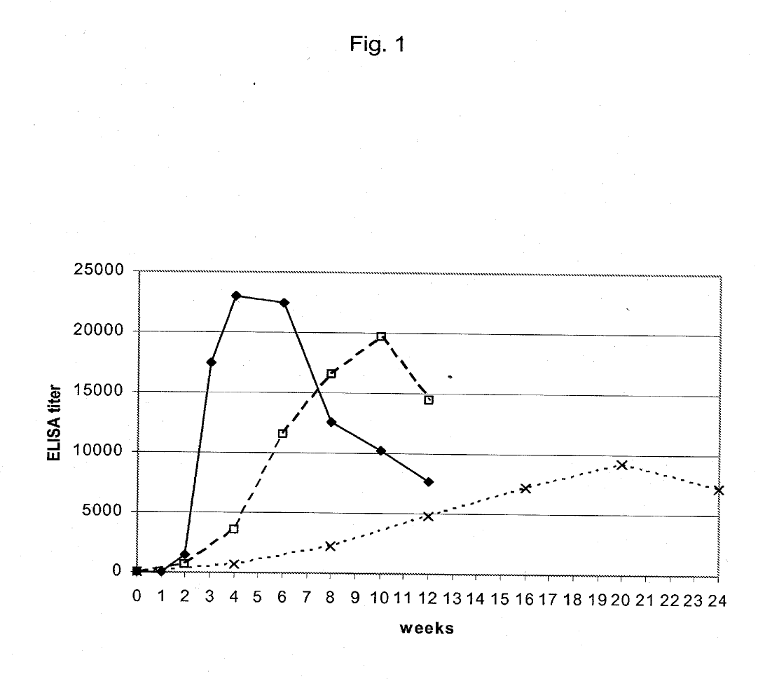

FIG. 1: Comparison of weekly, biweekly and monthly vaccination regimen:

Blood samples were collected at the time points as indicated in FIG. 1 for the measurement of anti-nicotine antibody titers to evaluate the immunogenicity of the particular dosing regimen. Anti-nicotine IgG antibody titer was determined by ELISA using well plates coated with RNAse-nicotine conjugate. The geometric mean values of antibody titers were shown in FIG. 1. The antibody binding affinity to nicotine was determined by equilibrium dialysis.

While with regimen 1 the highest antibody titer was reached at week 20, the peak was reached at week 10 and week 4 for regimen 2 and 3 respectively.

The highest antibody titers are 9000, 20000, and 23000 for regimen 1, 2 and 3 respectively. Thus the highest antibody titer achieved by regimen 3 and regimen 2 are about two times higher than that achieved by regimen 1.

At week 8 tested, all three regimens led to nicotine-specific antibodies with comparable affinity, which is around 50-100 nM.

### EXAMPLE 3

Regimen 4: Subject received five doses prepared as described in Example 1. The time interval between the first three administrations was 7 days followed by two administrations in 28 days intervals

Regimen 5: Subject received five doses prepared as described in Example 1. The time interval between the first four administrations was 7 days followed by one administration in a 28 days interval

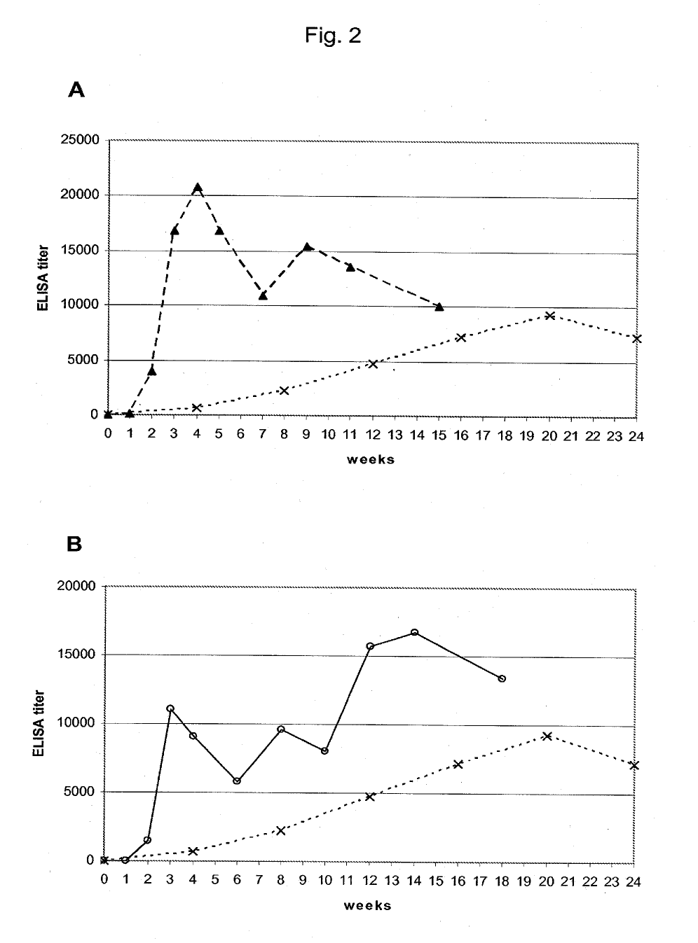

FIGS. 2A and 2B: Comparison of Regimen 4 and Regimen 5 and the monthly vaccination regimen:

Blood samples were collected at the time points as indicated in FIG. 2 for the measurement of anti-nicotine antibody titers to evaluate the immunogenicity of the particular dosing regimen. Anti-nicotine IgG antibody titer was determined by ELISA using well plates coated with RNAse-nicotine conjugate. The geometric mean values of antibody titers were shown in FIG. 2. The antibody binding affinity to nicotine was determined by equilibrium dialysis.

While with regimen 1 the highest antibody titer was reached at week 20, the peak was reached at week 4 for regimen 5. For regimen 4 an initial peak titer was reached at week 3, and the nicotine-specific IgG response was increased by the additional injections at week 6 and week 10.

The highest antibody titers after five injections were 9000, 21000, and 16000 for regimen 1, 4 and 5 respectively. Thus the highest antibody titer achieved by regimen 4 and regimen 5 are about two times higher than that achieved by regimen 1.

Regimen 4 and 5 are particularly advantageous since beside the early and rapid induction of high antibody titers, these regimens further ensure that the high antibody titers are maintained until week 8 or even until week 12. This is in particular beneficial taking into account that there is a higher danger of relapsing to smoking typically within the first 8-12 weeks.

At week 8 tested, all five regimens led to nicotine-specific antibodies with comparable affinity, with equilibrium dissociation values (Kd) of 50-100 nM.

1. A composition for prevention or treatment of addiction to a drug of abuse by inducing the drug of abuse-specific antibodies in a human, wherein said composition comprises:
(a) a virus-like particle of an RNA bacteriophage with at least one first attachment site,
(b) at least one drug of abuse with at least one second attachment site,
wherein (a) and (b) are covalently linked through said at least one first and said at least one second attachment site, and
wherein said composition is to be administered into said human, and wherein said administering of said composition into said human comprises at least a first, a second and a third administration of said composition into said human, wherein the time interval between said first administration and said second administration, and between said second administration and said third administration is at most 18 days.

2. The composition of claim 1, wherein said time interval between said first administration and said second administration, and between said second administration and said third administration is at most 14 days.

3. The composition of claim 1 or claim 2, wherein said time interval between said first administration and said second administration, and between said second administration and said third administration is 7 days.

4. The composition of any one of the preceding claims, wherein said administering of said composition into said human further comprises a fourth administration.

5. The composition of claim 4, wherein said administering of said composition into said human further comprises a fifth administration.

6. The composition of claim 5, wherein the time interval between the first administration and the second, the second and the third, the third and the fourth and the fourth and the fifth is 7 days.

7. The composition of any one of the claims 1-6, wherein the time interval between the third and the fourth and between the fourth and the fifth is longer than the time interval between the first and the second and is longer than the time interval between the second and the third.

8. The composition of claim 7, wherein the time interval between the first administration and the second, the second and the third is 7 days and the time interval between the third and the fourth and the fourth and the fifth is 28 days.

9. The composition of claim 4, wherein the time interval between said first and said second administration, said second and said third administration, and said third and said fourth administration is 7 days.

10. The composition of claim 5, wherein the time interval between said first and said second administration, said second and said third administration, and said third and said fourth administration is 7 days, and wherein the time interval between said fourth and said fifth administration is 28 days.

11. The composition of any one of the preceding claims, wherein said drug of abuse is nicotine.

12. The composition of any one of the preceding claims, wherein said disease is nicotine addiction.

13. The composition of any one of the preceding claims, wherein said composition comprises (a) a virus-like particle of an RNA bacteriophage Qβ and (b) at least one nicotine molecule, wherein the at least one nicotine molecule is covalently bound to the virus-like particle by a linking sequence.

14. The composition of claim 13, wherein said linking sequence consists of A-CH₂OCO(CH₂)₂CO—B, wherein A represents said nicotine molecule and wherein B represents said virus-like particle of RNA bacteriophage Qβ, and wherein said linking sequence is covalently bound to the 3′ position of said nicotine molecule.

15. A method for prevention or treatment of addiction to a drug of abuse by inducing the drug of abuse-specific antibodies in a human comprising administering into said human a composition comprising:
(a) a virus-like particle of an RNA bacteriophage with at least one first attachment site,
(b) at least one drug of abuse with at least one second attachment site,
wherein (a) and (b) are covalently linked through said at least one first and said at least one second attachment site, and
wherein said administering into said human of said composition comprises at least a first, a second and a third administration into said human of said composition, wherein the time interval between said first administration and said second administration, and between said second administration and said third administration is at most 18 days.

16. The method of claim 15, wherein said time interval between said first administration and said second administration, and between said second administration and said third administration is at most 14 days.

17. The method of claim 15 or claim 16, wherein said time interval between said first administration and said second administration, and between said second administration and said third administration is 7 days.

18. The method of any one of the claims 15-17 further comprising a fourth administration.

19. The method of claim 18 further comprising a fifth administration.

20. The method of claim 19, wherein the time interval between the first administration and the second, the second and the third, the third and the fourth and the fourth and the fifth is 7 days.

21. The method of any one of the claims 15-20, wherein the time interval between the third and the fourth and between the fourth and the fifth is longer than the time interval between the first and the second and is longer than the time interval between the second and the third.

22. The method of claim 21, wherein the time interval between the first administration and the second, the second and the third is 7 days and the time interval between the third and the fourth and the fourth and the fifth is 28 days.

23. The composition of claim 18, wherein the time interval between said first and said second administration, said second and said third administration, and said third and said fourth administration is 7 days.

24. The composition of claim 19, wherein the time interval between said first and said second administration, said second and said third administration, and said third and said fourth administration is 7 days, and wherein the time interval between said fourth and said fifth administration is 28 days.

25. The method of any one of the claims 15-24, wherein said drug of abuse is nicotine.

26. The method of any one of the claims 15-25, wherein said disease is nicotine addiction.

27. The composition of any one of the claims 15-26, wherein said composition comprises (a) a virus-like particle of an RNA bacteriophage Qβ and (b) at least one nicotine molecule, wherein the at least one nicotine molecule is covalently bound to the virus-like particle by a linking sequence.

28. The composition of claim 27, wherein said linking sequence consists of A-CH₂OCO(CH₂)₂CO—B, wherein A represents said nicotine molecule and wherein B represents said virus-like particle of RNA bacteriophage Qβ, and wherein said linking sequence is covalently bound to the 3′ position of said nicotine molecule.


SEQUENCE LISTING (19 sequences)

SEQ ID NO: 1 - protein, 132 aa, Enterobacteria phage Qbeta
AKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 2 - protein, 329 aa, Enterobacteria phage Qbeta
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAYWTLLIAGGGSGSKPDPVIPDPPIDPPPGTGKYTCPFAIWSLEEVYEPPTKNRPWPIYNAVELQPREFDVALKDLLGNTKWRDWDSRLSYTTFRGCRGNGYIDLDATYLATDQAMRDQKYDIREGKKPGAFGNIERFIYLKSINAYCSLSDIAAYHADGVIVGFWRDPSSGGAIPFDFTKFDKTKCPIQAVIVVPRA

SEQ ID NO: 3 - protein, 129 aa, Enterobacteria phage R17
ASNFTQFVLVNDGGTGNVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 4 - protein, 130 aa, Enterobacteria phage fr
MASNFEEFVLVDNGGTGDVKVAPSNFANGVAEWISSNSRSQAYKVTCSVRQSSANNRKYTVKVEVPKVATQVQGGVELPVAAWRSYMNMELTIPVFATNDDCALIVKALQGTFKTGNPIATAIAANSGIY

SEQ ID NO: 5 - protein, 130 aa, Enterobacteria phage GA
MATLRSFVLVDNGGTGNVTVVPVSNANGVAEWLSNNSRSQAYRVTASYRASGADKRKYAIKLEVPKIVTQVVNGVELPGSAWKAYASIDLTIPIFAATDDVTVISKSLAGLFKVGNPIAEAISSQSGFYA

SEQ ID NO: 6 - protein, 132 aa, Enterobacteria phage SP
MAKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAY

SEQ ID NO: 7 - protein, 329 aa, Enterobacteria phage SP
AKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAYWAALLVASSGGGDNPSDPDVPVVPDVKPPDGTGRYKCPFACYRLGSIYEVGKEGSPDIYERGDEVSVTFDYALEDFLGNTNWRNWDQRLSDYDIANRRRCRGNGYIDLDATAMQSDDFVLSGRYGVRKVKFPGAFGSIKYLLNIQGDAWLDLSEVTAYRSYGMVIGFWTDSKSPQLPTDFTQFNSANCPVQTVIIIPS

SEQ ID NO: 8 - protein, 130 aa, Enterobacteria phage MS2
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 9 - protein, 133 aa, Enterobacteria phage M11
MAKLQAITLSGIGKKGDVTLDLNPRGVNPTNGVAALSEAGAVPALEKRVTISVSQPSRNRKNYKVQVKIQNPTSCTASGTCDPSVTRSAYSDVTFSFTQYSTVEERALVRTELQALLADPMLVNAIDNLNPAY

SEQ ID NO: 10 - protein, 133 aa, Enterobacteria phage MX1
MAKLQAITLSGIGKNGDVTLNLNPRGVNPTNGVAALSEAGAVPALEKRVTISVSQPSRNRKNYKVQVKIQNPTSCTASGTCDPSVTRSAYADVTFSFTQYSTDEERALVRTELKALLADPMLIDAIDNLNPAY

SEQ ID NO: 11 - protein, 330 aa, Enterobacteria phage NL95
MAKLNKVTLTGIGKAGNQTLTLTPRGVNPTNGVASLSEAGAVPALEKRVTVSVAQPSRNRKNYKVQIKLQNPTACTKDACDPSVTRSGSRDVTLSFTSYSTERERALIRTELAALLKDDLIVDAIDNLNPAYWAALLAASPGGGNNPYPGVPDSPNVKPPGGTGTYRCPFACYRRGELITEAKDGACALYACGSEALVEFEYALEDFLGNEFWRNWDGRLSKYDIETHRRCRGNGYVDLDASVMQSDEYVLSGAYDVVKMQPPGTFDSPRYYLHLMDGIYVDLAEVTAYRSYGMVIGFWTDSKSPQLPTDFTRFNRHNCPVQTVIVIPSL

SEQ ID NO: 12 - protein, 129 aa, Enterobacteria phage f2
ASNFTQFVLVNDGGTGNVTVAPSNFANGVAEWISSNSRSQAYKVTCSVRQSSAQNRKYTIKVEVPKVATQTVGGVELPVAAWRSYLNLELTIPIFATNSDCELIVKAMQGLLKDGNPIPSAIAANSGIY

SEQ ID NO: 13 - protein, 128 aa, Enterobacteria phage PP7
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 14 - protein, 131 aa, Enterobacteria phage AP205
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 15 - protein, 132 aa, Artificial Sequence
  Description of Artificial Sequence Synthetic Enterobacteria phage Qbeta 240 mutant
AKLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 16 - protein, 132 aa, Artificial Sequence
  Description of Artificial Sequence Synthetic Enterobacteria phage Q-beta 243 mutant
AKLETVTLGKIGKDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 17 - protein, 132 aa, Artificial Sequence
  Description of Artificial Sequence Synthetic Enterobacteria phage Q-beta 250 mutant
ARLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 18 - protein, 132 aa, Artificial Sequence
  Description of Artificial Sequence Synthetic Enterobacteria phage Q-beta 251 mutant
AKLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 19 - protein, 132 aa, Artificial Sequence
  Description of Artificial Sequence Synthetic Enterobacteria phage Q-beta 259 mutant
ARLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY